(19) United States
(12) Patent Application Publication
(10) Pub. No.: US 2026/0215668 A1
(43) Pub. Date: Jul. 30, 2026

(54) REMOVABLE IMAGING SYSTEM FOR MEDICAL SYSTEM

(71) Applicant: Boston Scientific Scimed, Inc., Maple Grove, MN (US)

(72) Inventor: Richard C. TAH, Milford, MA (US)

(73) Assignee: Boston Scientific Scimed, Inc., Maple Grove, MN (US)

(21) Appl. No.: 19/443,241

(22) Filed: Jan. 8, 2026

Related U.S. Application Data
(60) Provisional application No. 63/743,698, filed on Jan. 10, 2025.

(51) Int. Cl. A61B 1/05; A61B 1/00; A61B 1/018; A61B 1/06; A61B 18/04; A61B 18/00

(52) U.S. Cl. CPC A61B 1/053; A61B 1/00066; A61B 1/00114; A61B 1/00119; A61B 1/00124; A61B 1/00128; A61B 1/00137; A61B 1/018; A61B 1/0676; A61B 18/04; A61B 2018/00178; A61B 2018/00577; A61B 2018/0091; A61B 2018/00982; A61B 2018/048

(57) ABSTRACT

A medical system may include a medical device and an insertion portion extending from the handle, the insertion portion having a first lumen and a second lumen, a removable camera sheath, and a needle extending through the first lumen. The removable camera sheath may be configured to removably extend through the second lumen. The removable camera sheath may include a shaft and a connector assembly. The shaft may have a proximal end and a distal end. The distal end may have an imaging device and a lighting element. The connector assembly may be coupled to the proximal end of the shaft. The connector assembly may include a keying feature configured to engage with a receiving feature of the handle.

### CROSS-REFERENCE TO RELATED APPLICATIONS

This application claims the benefit of priority from U.S. Provisional Application No. 63/743,698, filed on January 10, 2025, which is incorporated by reference herein in its entirety.

### FIELD OF INVENTION

The disclosure relates generally to medical devices incorporating imaging capabilities, and more particularly to a removable camera sheath configured to provide visualization for use with a medical device during minimally invasive procedures involving vapor delivery.

### BACKGROUND

Certain medical conditions, such as conditions of the prostate, may be treated by ablation, including by vapor ablation. Such ablation may be performed using a device having a sheath that is inserted into a body lumen or otherwise into a body of a patient. The sheath may include a catheter (e.g., a needle) that may be deployed in order to deliver vapor or another type of ablative energy. In ablation procedures, visualization of the area being treated may assist in performing the procedure effectively. The systems, devices, and methods of the disclosure may facilitate such visualization.

### SUMMARY

According to an example, a medical system may include a medical device and an insertion portion extending from the handle, the insertion portion having a first lumen and a second lumen, a removable camera sheath, and a needle extending through the first lumen. The removable camera sheath may be configured to removably extend through the second lumen. The removable camera sheath may include a shaft and a connector assembly. The shaft may have a proximal end and a distal end. The distal end may have an imaging device and a lighting element. The connector assembly may be coupled to the proximal end of the shaft. The connector assembly may include a keying feature configured to engage with a receiving feature of the handle.

Any of the devices disclosed herein may include any of the following features, alone or in any combination. The keying feature may extend away from a central longitudinal axis of the connector assembly. The handle may include a receiving channel configured to receive the keying feature. The receiving channel may include a recessed portion having a complementary shape of the keying feature. The receiving channel may be configured to accommodate the keying feature. The connector assembly may include a keying surface adjacent to the keying feature. The handle may include a complementary surface configured to engage with the keying surface.

The connector assembly may include a casing having a first casing portion and a second casing portion. The connector assembly may further include a strain relief disposed between the casing and the shaft. The strain relief may include a rib configured to engage with a cutout of the shaft.

The receiving channel may include one or more protrusions or flanges extending laterally inward from a surface of the receiving channel. The one or more flanges may be shaped to receive the keying feature of the connector assembly. The one or more flanges may be configured to at least partially surround the keying feature.

A distal end of the removable camera sheath may be angled by at least forty degrees relative to a longitudinal axis of the removable camera sheath. The insertion portion may include a distal opening disposed on a radially outer surface of the insertion portion. The needle may be configured to extend through the distal opening. The distal end of the removable camera sheath may terminate proximally of the distal opening.

The medical system may further comprise a controller and a connecting element configured to electronically couple the removable camera sheath to the controller. The connector assembly may include a receiver configured to removably receive the connecting element. The connector assembly may further include a board electrically coupled to the imaging device and the lighting element. The board may be configured to transmit data between the imaging device and the controller via the connecting element.

According to another example, a medical system may include a medical device having a handle and an insertion portion extending from the handle and a removable sheath. The insertion portion may have at least a first lumen and a second lumen. The handle may define a receiving channel. The removable camera sheath may be configured to removably extend through the second lumen. The removable camera sheath may include a shaft having a proximal end and a distal end, and a connector assembly coupled to the proximal end of the shaft. The distal end of the shaft may include an imaging device and a lighting element. The connector assembly may include a keying feature. The receiving channel may include one or more flanges extending laterally inward from a surface of the receiving channel. The one or more flanges may be configured to receive the keying feature of the connector assembly.

Any of the devices disclosed herein may include any of the following features, alone or in any combination. The one or more flanges may be configured to at least partially surround the keying feature. The connector assembly may include a strain relief having a rib. The shaft may include a cutout configured to receive the rib to maintain proper orientation of the shaft relative to the strain relief. The rib may be integrally formed with the strain relief. The rib may extend radially inward from an inner surface of the strain relief.

According to another example, a camera sheath may be configured for insertion and removal from a medical device. The camera sheath may comprise a shaft and a connector assembly. The shaft may have a proximal end and a distal end. The distal end may have an imaging device and a lighting element. The connector assembly may be coupled to the proximal end of the shaft. The connector assembly may comprise a keying feature. The keying feature may be configured to engage with a receiving feature of the medical device. The keying feature may include a cylindrical body extending away from a longitudinal axis of the connector assembly.

Any of the devices disclosed herein may include the following feature. The distal end of the shaft may be angled by at least forty degrees relative to a longitudinal axis of the shaft.

The foregoing general description of the illustrative embodiments and the following detailed description thereof are merely exemplary aspects of the teachings of this disclosure and are not restrictive.

### BRIEF DESCRIPTION OF FIGURES

The accompanying drawings, which are incorporated and constitute a part of this specification, illustrate various exemplary embodiments and, together with the description, serve to explain the principles of the disclosed embodiments.

FIG. 1 illustrates a side view of a medical system, according to aspects of the disclosure.

FIG. 2A illustrates an exploded view of a removable sheath for the medical system of FIG. 1, according to embodiments.

FIG. 2B illustrates a perspective view of the removable sheath of FIG. 2A, according to aspects of the disclosure.

FIG. 3A illustrates an exploded view of a distal tip of the removable sheath of FIGS. 2A and 2B, according to embodiments.

FIG. 3B illustrates a side view of the distal tip FIG. 3A.

FIG. 4 illustrates a sectional view of the removable sheath of FIGS. 2A and 2B, according to embodiments.

FIG. 5A illustrates a perspective view of an assembly of the medical system of FIG. 1 in a first configuration, according to aspects of the disclosure.

FIG. 5B illustrates a perspective view of the assembly of FIG. 5A in a second configuration, according to an embodiment.

### DETAILED DESCRIPTION

It may be understood that both the foregoing general description and the following detailed description are exemplary and explanatory only and are not restrictive of the invention, as claimed. As used herein, the terms “comprises,” “comprising,” “includes,” “including,” “has,” “having,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “distal” refers to a direction away from an operator, and the term “proximal” refers to a direction toward an operator. In some drawings, arrows labeled “P” and “D” indicate proximal and distal directions, respectively. The term “exemplary” is used in the sense of “example,” rather than “ideal.” The term “approximately,” or like terms (e.g., “substantially”), includes values +/- 10% of a stated value.

Medical devices incorporating imaging capabilities sometimes integrate cameras directly into the device structure. Such devices may implicate complicated design considerations. The disclosure relates to a medical system that integrates imaging capabilities with a needle delivery system. The system may include a medical device with a removable camera sheath, allowing for visualization during minimally invasive procedures. Various embodiments of the system may incorporate features such as fluid delivery, suction capabilities, and ergonomic design elements to enhance functionality and user experience.

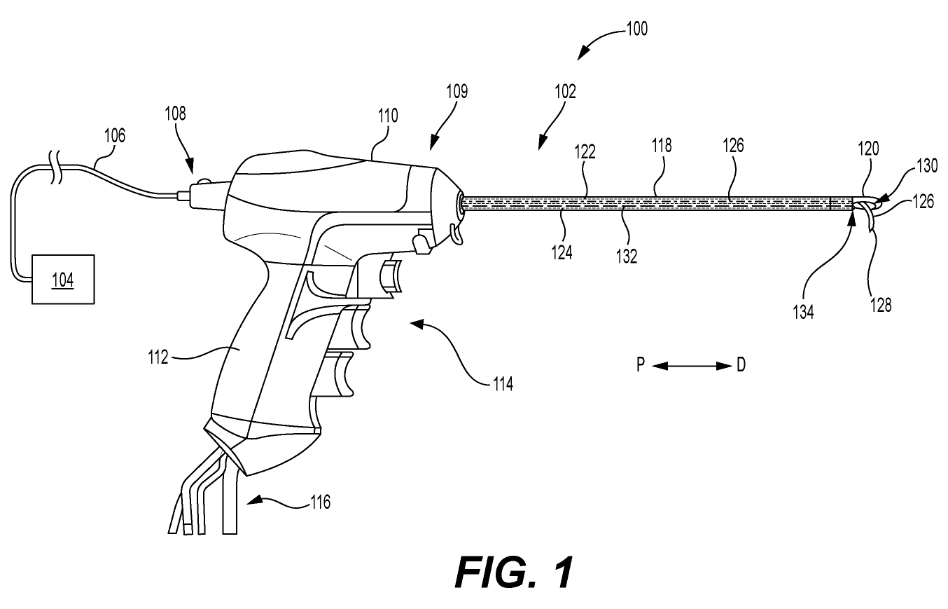

FIG. 1 illustrates a side view of a medical system 100, according to aspects of the disclosure. Medical system 100 may include a medical assembly 102, a controller 104, and a connecting element 106 (e.g., umbilicus or other type of cabling) electronically coupling medical assembly 102 to controller 104.

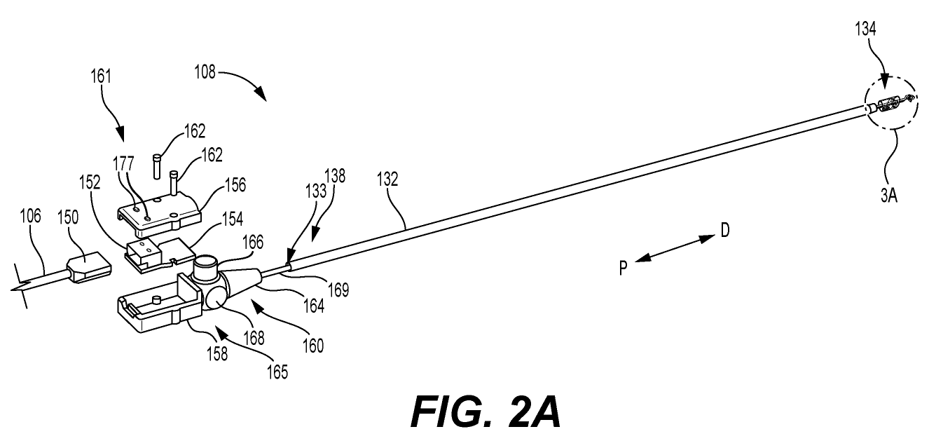

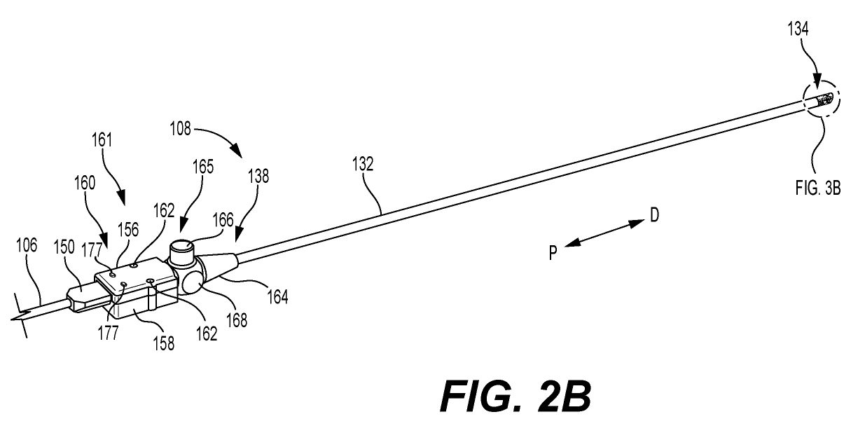

The medical assembly 102 may include a removable camera sheath 108 configured to releasably couple to a medical device 109 The removable nature of the camera sheath 108 provides flexibility in device configuration, allowing for easy cleaning, sterilization, or replacement of imaging components. In aspects, camera sheath 108 and medical device 109 may be disposable. In alternative aspects, camera sheath 108 may be configured for multiple uses and medical device 109 may be configured for a single use, or vice-versa. Aspects of removable camera sheath 108, including how removable camera sheath 108 may be coupled to medical device 109, are described in further detail with respect to FIGS. 2A-5B.

Referring still to FIG. 1, medical device 109 may include a handle 110 and an insertion portion 118 (e.g., a shaft). Handle 110 may include a gripping portion 112 configured for gripping by a user during a procedure. In some aspects, handle 110 may be configured similar to a pistol grip, although other configurations (e.g., a straight grip, etc.) may also be used. The handle 110 may be equipped with actuators 114 for controlling various functions of the device, such as, for example, the application of suction, delivery of fluids, delivery of air, actuation or delivery of a tool (e.g., a needle 126, discussed below), etc. A position of actuators 114 is merely exemplary, and one or more of actuators 114 may be located in any suitable position, including elsewhere on handle 110. One or more fluidics tubes and cables 116 may extend from handle 110, for example, to a fluid and/or suction source (not shown) and to couple medical device 109 to power and/or other electronic elements.

Medical device 109 may include insertion portion 118 extending from a distal end of handle 110. In some aspects, insertion portion 118 may be an elongated structure designed for insertion into a patient's body (e.g., into a patient’s urethra) during medical procedures. Insertion portion 118 may terminate in a distal tip 120, which may be configured for atraumatic insertion and navigation within bodily cavities or tissues. The length and diameter of insertion portion 118 may vary depending on the specific medical application and patient anatomy. In some aspects, insertion portion 118 may be rigid, while in other aspects, insertion portion 118 may be flexible or semi-flexible to allow for maneuverability within the body.

Insertion portion 118 may include one or more internal lumens extending along its length. For example, insertion portion 118 may include a first lumen 122 and a second lumen 124. These lumens may serve various purposes, such as providing channels for fluid delivery, suction, and/or medical instruments, such as a portion of camera sheath 108 and needle 126. For example, needle 126 may extend through at least a portion of first lumen 122, and a shaft 132 of camera sheath 108 may be removably extendable through at least a portion of second lumen 124. Shaft 132 may comprise, for example, plastic or metal and may be sufficiently stiff so as to allow shaft 132 to be extended through second 124. For example, shaft 132 may be rigid.

Accommodation of shaft 132 of camera sheath 108 in second lumen 124 allows for integration of imaging capabilities with other medical functions. This design feature enables medical assembly 102 to combine visual guidance with therapeutic or diagnostic capabilities in a single instrument. Second lumen 124 may incorporate features (e.g., rails, protrusions, indentations, etc.) to ensure proper alignment and secure attachment of shaft 132 of camera sheath 108 within second lumen 124, and to assist with maintaining image quality and stability during medical procedures.

In some aspects, needle 126 may be a hollow, elongated structure configured to deliver vapor to a tissue (e.g., to a tissue of a prostate). Needle 126 may terminate in a needle tip 128 at its distal end. A distal opening 130 may be present at distal tip 120 of insertion portion 118, through which needle tip 128 may protrude (e.g., radially outward relative to insertion portion 118). Distal opening 130 may be positioned on a side surface of the insertion portion 118, allowing for lateral (radially outward) deployment of needle 126. The size and shape of distal opening 130 may be designed to accommodate needle 126 while maintaining a smooth external profile of insertion portion 118 when needle 126 is retracted. For example, needle 126 may be retractable, allowing it to be withdrawn into first lumen 122 when not in use. In some aspects, one or more of actuators 114 of handle 110 may be configured for extending or retracting needle 126.

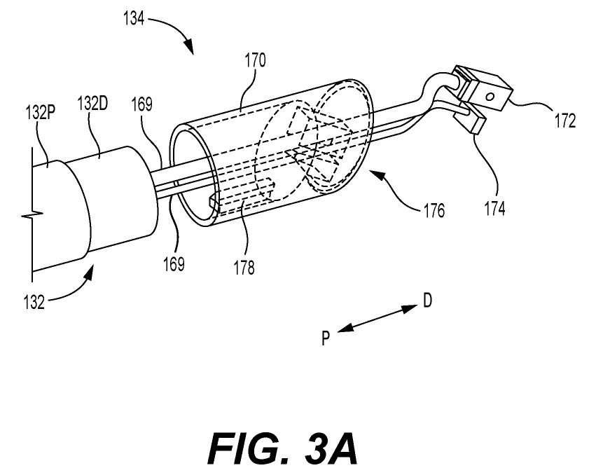

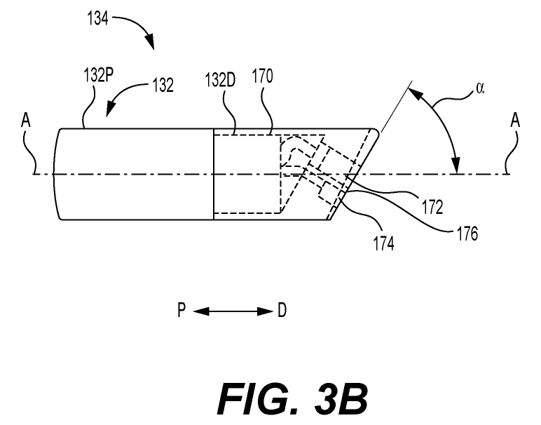

In aspects, distal opening 130 may define a distal opening for both first lumen 122 and second lumen 124. This configuration may allow for the deployment of instruments or delivery of fluids (e.g., cooling fluids) from first lumen 122 and/or second lumen 124 through a single opening at the distal end of the device. Furthermore, distal opening 130 may allow for visualization of needle tip 128 by an imaging device 172 disposed on a distalmost end of shaft 132 of camera sheath 108 (shown in detail in FIGS. 3A and 3B). The positioning of distal opening 130 at distal tip 120, for example, in combination with imaging device 172 disposed at a distal end of shaft 132, may enable precise control over the direction and depth of needle tip 128 and/or fluid delivery therefrom during medical procedures. The distalmost end of shaft 132 may terminate proximally of distal opening 130.

In some cases, needle tip 128 may be biased to be angled relative to a longitudinal axis of insertion portion 118 (e.g., in an extended state). Additionally, or alternatively, distal tip 120 may include internal features (not shown) such that, as needle 126 is extended distally relative to insertion portion 118, needle tip 128 is angled relative to a longitudinal axis of insertion portion 118.

Insertion portion 118 may include external features along its length. These may include depth markings to assist in determining insertion distance. In some aspects, insertion portion 118 may include features to enable visualization of insertion portion 118 under medical imaging. For example, insertion portion 118 may incorporate radiopaque markers or materials at strategic locations along its length. These radiopaque elements may be visible under imaging modalities such as X-ray, fluoroscopy, or computed tomography (CT), allowing healthcare providers to track the position and orientation of insertion portion 118 within a patient's body during procedures. In some cases, insertion portion 118 may be constructed from or coated with materials that enhance its visibility under ultrasound imaging. Additionally, insertion portion 118 may include patterns or textures on its surface that create distinctive echogenic signatures, further improving its visibility and localization during ultrasound-guided procedures.

Controller 104 may house the main processing unit that coordinates various functions of medical system 100. This may include managing power distribution, processing user inputs, controlling imaging and lighting elements of camera sheath 108, controlling a vapor generator of handle 110, and providing fluid (e.g., ablation fluid or cooling fluid) to medical device 109, among other aspects. Controller 104 may have data storage capabilities for recording and archiving video feeds and procedural data. In some aspects, controller 104 may include wireless connectivity options to enable integration with hospital information systems or remote consultation capabilities. In some cases, controller 104 may include a monitor or display device for presenting visual information to a user. The monitor may display live video feeds from camera sheath 108, as well as other relevant information such as patient data, device settings, or procedural guidelines.

Controller 104 may incorporate touch screen functionality in some aspects, allowing users to interact with the displayed information and control various aspects of medical assembly 102. Controller 104 may also include image processing capabilities to enhance, analyze, or manipulate the video feed from camera sheath 108 in real-time.

Connecting element 106 may be an HDMI cable or another suitable data (e.g., video) transmission device. In some aspects, connecting element 106 may be configured to transmit information from camera sheath 108 to controller 104. This information may include video data, image data, sensor readings, or other relevant data collected by components within camera sheath 108. In some cases, connecting element 106 may be removably connected to one or both ends of medical system 100. For example, connecting element 106 may be removably connected to controller 104 and/or camera sheath 108. This removable connection may allow for easy replacement, upgrading, or maintenance of the connecting element without requiring replacement of the entire system. The removable nature of the connection may also facilitate cleaning and sterilization procedures of connecting element 106 and, optionally, reuse of connecting element 106. In other aspects, connecting element 106 may be fixedly coupled to one of controller 104 or camera sheath 108. With connecting element 106 fixedly coupled to camera sheath 108, connecting element 106 (and camera sheath 108) may be removable relative to handle 110.

Connecting element 106 may incorporate various types of connectors at its ends to ensure secure and reliable connections. These connectors may include standard interfaces such as HDMI, USB, or proprietary connectors designed specifically for medical system 100. In some aspects, connecting element 106 may include features to prevent accidental disconnection during use, such as locking mechanisms or threaded connectors. The length of connecting element 106 may vary depending on the specific requirements of the medical procedure and the desired positioning of controller 104 relative to the patient and medical personnel. In some cases, connecting element 106 may include one or more intermediate connection points or adapters to allow for customization of its length or to facilitate routing through complex medical environments.

FIG. 2A illustrates an exploded view of removable camera sheath 108, and FIG. 2B illustrates an assembled view of removable camera sheath 108. As described above with respect to FIG. 1, removable camera sheath 108 may include a shaft 132. Shaft 132 may have a lumen 133 extending therethrough. A distal end 134 of shaft 132 is shown and described below with respect to FIGS. 3A and 3B. A connector assembly 161 may be coupled to a proximal end 138 of shaft 132. Connector assembly 161 may be configured to assist with aligning camera sheath 108 within handle 110 of medical device 109 and coupling camera sheath 108 to handle 110.

Connector assembly 161 may include a casing 160 comprising a first casing portion 156 and a second casing portion 158. In some cases, fastener(s) 162 may secure first casing portion 156 to second casing portion 158. In some cases, fastener 162 may be a screw, bolt, or other type of mechanical fastener configured to couple first casing portion 156 to second casing portion 158. Additionally or alternatively, first casing portion 156 may be coupled to second casing portion 158 using an adhesive, press fit connection, snap fit connection, ultrasonic welding, or other attachment methods commonly used in the art. A distal end of connector assembly 161 may include a strain relief 164. Strain relief 164 may help prevent damage to wires, cables, or other elements extending through lumen 133 of shaft 132.

Connector assembly 161 may include a central portion 165 (e.g., disposed axially between casing 160 and strain relief 164). Central portion 165 may include a keying feature 166 extending away from a longitudinal axis of central portion 165/connector assembly 161. For example, keying feature 166 may extend upward from adjacent portions of connector assembly 161 in a configuration in which connector assembly 161 is coupled to handle 110. In aspects, keying feature 166 may be a cylindrical body. However, keying feature 166 may be any shape and/or size. For example, keying feature 166 may be cylindrical (as shown), cuboidal, pyramidal, etc. In some examples, top and bottom ends of keying feature 166 may have flanges.

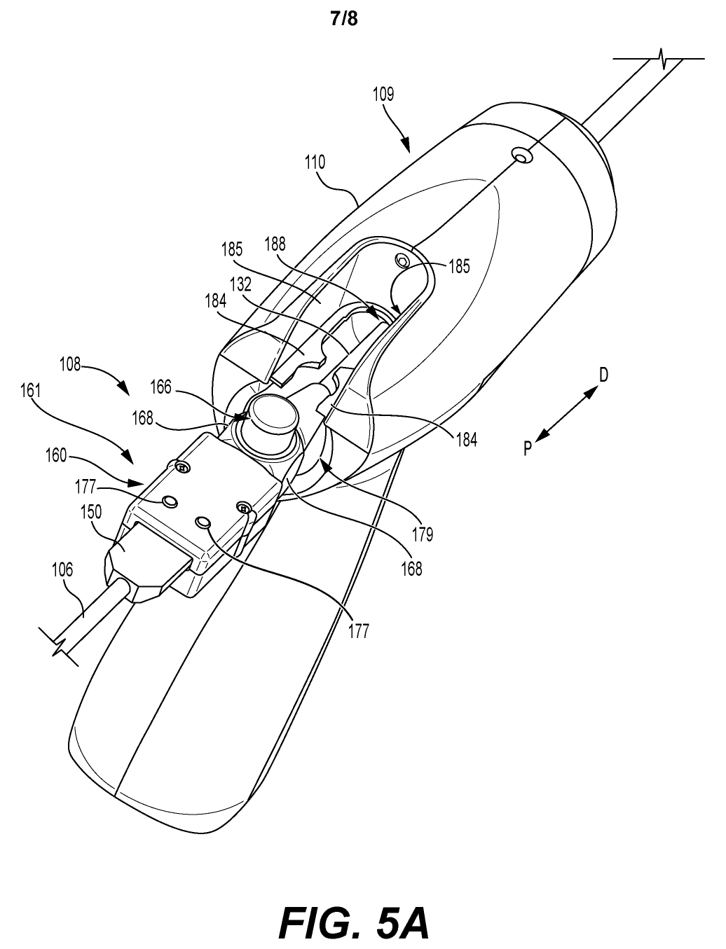

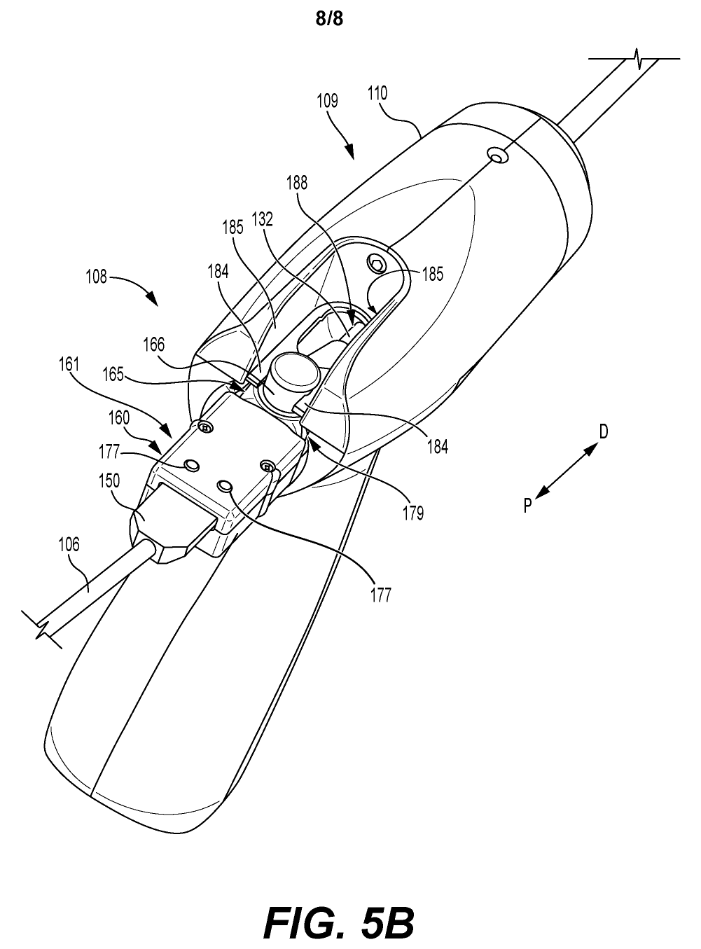

Central portion 165 may further include one or more keying surfaces 168, which may not be visible in the assembled state (e.g., FIG. 5B). In some cases, keying feature 166 and keying surface 168 may assist with proper alignment of removable camera sheath 108 and handle 110, as will be described in more detail below with respect to FIGS. 5A and 5B. Keying surface(s) 168 may be planar surfaces that are adjacent to keying feature(s) 166. For example, keying surface(s) 168 may be on lateral sides of central portion 165 when connector assembly 161 is coupled to handle 110. In aspects, keying surface(s) 168 may include flat, approximately circular faces. In some aspects, keying surface(s) 168 may include one or more securing features (e.g., ridges, detents, indents, etc.). Internal surfaces of handle 110 may include complementary features configured to engage such securing features on keying surface(s) 168. For example, such securing features may assist in locking or coupling camera sheath 108 to handle 110.

Connecting element 106 may extend proximally from a connector 150. In some cases, as discussed above, connecting element 106 may provide power and/or data transmission capabilities between imaging device 172 of distal end 134 and external components. Connector 150 may be an HDMI connector, a USB connector, or any other type of connector commonly used for transmitting information electronically. Alternatively, connector 150 may be a custom-fit connector.

In some aspects, first casing portion 156 and second casing portion 158 may together define casing 160 for housing one or more electrical components. For example, the hollowed portion may be configured to hold a receiver 152 and an electrical board 154 (e.g., circuit board). Receiver 152 may be configured to removably receive an end of connector 150, allowing for transmission of power and/or data between board 154 and controller 104, e.g., via connecting element 106.

At least one wire or cable 169 may extend distally from casing 160. The at least one wire or cable 169 may include one or more of an imaging cable/wire (e.g., coupled to imaging device 172 and/or lighting cable/wire (coupled to lighting element 174, discussed below). A proximal end of the at least one wire or cable 169 may be electrically fixed to board 154. The at least one wire or cable 169 may be configured to transmit power and/or data to/from imaging device 172 and lighting element 174 to board 154. When assembled, first casing portion 156 and second casing portion 158 may fully enclose board 154 within casing 160. An opening of receiver 152 may be disposed on a proximal-most end of casing 160, thus facilitating the attachment or removal of connector 150 from receiver 152.

In some aspects, one or more actuators 177 (e.g., buttons, switches, etc.) may be disposed on one or more surfaces of connector assembly 161casing160 (e.g., on a top surface of first casing portion 156). Actuators 177 may be configured to control aspects of imaging device 172 and/or lighting element 174. For example, actuator(s) 177 may control power to imaging device 172 and/or lighting element 174, capture images or videos, adjust brightness levels of lighting element 174, modify focus or zoom settings of imaging device 172, or initiate data transmission between camera sheath 108 and controller 104. Although actuator(s) 177 may be positioned anywhere on connector assembly 161, in some cases, actuator(s) 177 may be positioned on surfaces of connector assembly 161 that are easily accessible to a user during operation of medical system 100.

FIGS. 3A and 3B illustrate a distal end 134 of camera sheath 108. FIG. 3A shows an exploded view of distal end 134, while FIG. 3B shows an assembled view of distal end 134. Distal end 134 may include a cap 170 housing an imaging device 172 and one or more lighting elements 174. In some aspects, distal end 134 may include internal components shown in broken lines in FIGS. 3A and 3B. The broken lines may indicate features or components that are disposed or defined within cap 170 or shaft 132. For example, imaging device 172 and lighting element 174 are shown in broken lines in FIG. 3B to indicate their position within cap 170 (e.g., in an assembled state). In some cases, imaging cable 169A and lighting cable 169B may also be shown in broken lines to indicate their routing through cap 170.

Referring to both FIG. 3A and 3B, cap 170 may be coupled to a distal end of shaft 132. Cap 170 may include a cap keying feature 178 configured to align with a corresponding feature on a distal end of shaft 132, ensuring proper orientation of cap 170 with shaft 132, for example, during assembly. In some cases, cap keying feature 178 may comprise a protrusion extending radially inward from an inner surface of cap 170 or a notch that extends radially inward, relative to the inners surface of cap 170. Cap keying feature 178 may mate with a complementary feature (e.g., recess or protrusion) on shaft 132. In aspects, the protrusion may have a generally rectangular or trapezoidal shape when viewed from the side. When viewed proximally to distally (or vice-versa), cap keying feature 178 may also have a rectangular or trapezoidal shape, or a circular or semi-circular shape, a T-shape, etc. In some aspects, cap keying feature 178 may be an elongated ridge extending longitudinally along an internal portion of cap 170. The ridge may have a complementary shape to a protrusion on shaft 132.

In some aspects, cap keying feature 178 may include multiple protrusions and/or recesses arranged in a specific pattern to ensure unique alignment with shaft 132. The size and shape of cap keying feature 178 may be designed to provide a secure fit and prevent rotation between cap 170 and shaft 132 when assembled.

A distalmost end of shaft 132 may extend at least partially through a proximal end of cap 170. In these aspects, a diameter of a distalmost end 132D of shaft 132 may be less than a diameter of a more proximal portion 132P of shaft 132.

In some aspects, for example, when cap 170 is coupled to shaft 132, a radially outer surface of cap 170 and a radially outer surface of shaft 132 may form a continuous surface. Accordingly, an outer diameter of cap 170 may be equal to an outer diameter of shaft 132 (e.g., proximal to the distalmost end of shaft 132 that extends through the proximal end of cap 170). This configuration may provide a smooth transition between shaft 132 and cap 170, which may facilitate insertion and removal of camera sheath 108 from medical device 109 during medical procedures.

An imaging device 172 (e.g., a camera) and one or more lighting elements 174 (e.g., light emitting diodes (LEDs)) may be disposed on a distalmost face 176 of cap 170. Imaging device 172 may be configured to capture images and/or video during medical procedures. As shown more clearly in FIG. 3B, distalmost face 176 of cap 170 may be angled relative to a longitudinal axis A, of shaft 132. For example, distalmost face 176 may have a normal line that is not parallel to the longitudinal axis of shaft 132. The angle of distalmost face 176 may be approximately 40-80 degrees or, more specifically, approximately 60 degrees (e.g., relative to longitudinal axis A of shaft 132). In some cases, the angled orientation may facilitate visualization of medical instruments or procedures performed using medical system 100. For example, distalmost face 176 may be angled so as to facilitate the visualization of needle 126 being extended out of (or retracted into) distal opening 130, as shown in FIG. 1. In aspects, distalmost face 176 of cap 170 may terminate adjacent to or proximal of distal opening 130.

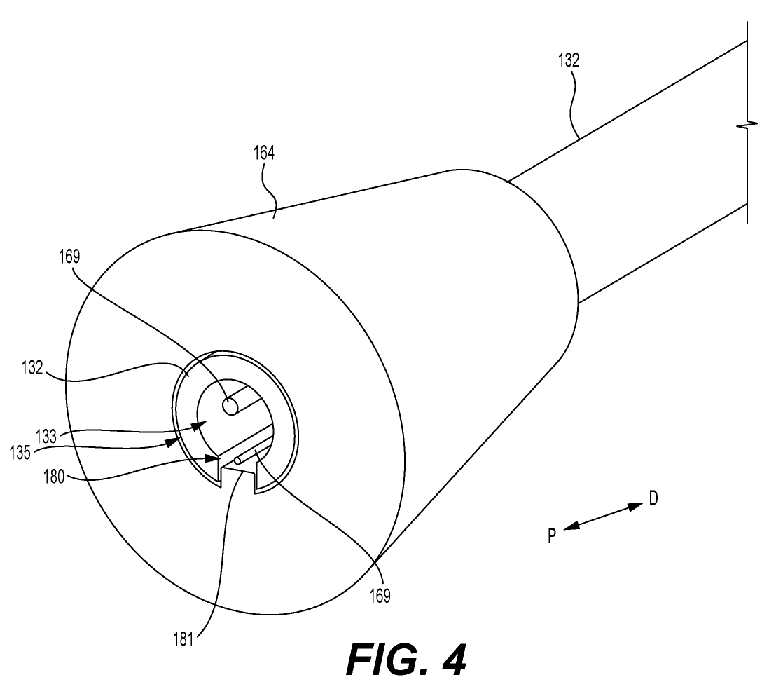

FIG. 4 illustrates a transverse cross-sectional view of a strain relief 164 of camera sheath 108. For example, the cross-sectional view of strain relief 164 is perpendicular to the longitudinal axis of shaft 132.

Strain relief 164 may include a lumen 135 configured to receive a proximal end of shaft 132. In some aspects, a proximal end of shaft 132 may include a cutout 180 (e.g., an opening) configured to receive a rib 181 of strain relief 164. Rib 181 may assist with alignment of shaft 132 within strain relief 164. The engagement between cutout 180 and rib 181 may help maintain proper orientation of shaft 132 relative to strain relief 164 during assembly and use of camera sheath 108. In some cases, strain relief 164 may include additional features to secure shaft 132, such as internal grooves, channels, or protrusions that correspond to external features of shaft 132.

Rib 181 may extend radially inward from an inner surface of the strain relief, for example, towards the central longitudinal axis of shaft 132. Rib 181 may be integrally formed with strain relief 164. For example, rib 181 may be a feature made or molded with strain relief 164. In other aspects, rib 181 may be a separate component fixed to strain relief 164. For example, rib 181 may be attached to strain relief 164 using adhesives, mechanical fasteners, welding, or other attachment methods commonly used in the art. In some cases, rib 181 may be made from a different material than strain relief 164. For example, rib 181 may be formed from a rigid material to provide enhanced structural support and alignment capabilities, while strain relief 164 may be formed from a more flexible material to accommodate bending and movement of shaft 132.

FIGS. 5A and 5B illustrate perspective views of a medical assembly 102 in an unassembled state (FIG. 5A) and an assembled state (FIG. 5B), respectively.

Handle 110 may include a receiving channel 179 configured to receive a portion of camera sheath 108. For example, distal end 134 of camera sheath 108 may be inserted into receiving channel 179 and extended into and through second lumen 124 until connector assembly 161 is within receiving channel 179. In some aspects, receiving channel 179 may be shaped to mate with the proximal portion of camera sheath 108. For example, the shape of receiving channel 179 may include contours and dimensions that correspond to the external profiles of casing 160, central portion 165, and strain relief 164. In some cases, receiving channel 179 may include recessed portions or protrusions that align with corresponding features on connector assembly 161 to ensure proper positioning and secure attachment during assembly of medical assembly 102. In particular, receiving channel 179 may include one or more receiving features 184 configured to engage with corresponding features of camera sheath 108. For example, receiving features 184 may be configured to engage with keying feature 166 and keying surface 168 of camera sheath 108 to ensure proper alignment of camera sheath 108 relative to handle 110 during assembly.

In aspects, receiving features 184 may be comprised of one or more protrusions or flanges, each extending inwards (e.g., laterally or radially inward, toward a central longitudinal axis of channel 179) from a surface 185 of channel 179. For example, receiving features 184 may be protrusions or flanges extending from surface 185 and inwards, or towards, a longitudinal axis of handle 110. In aspects, keying feature 166 may include one or more grooves or slots to accommodate receiving features 184. Receiving features 184 may be configured to flex to accommodate keying feature 166.

In some aspects, receiving channel 179 may be continuous with a thru hole 188, which may extend distally through handle 110. Thru hole 188 may be coaxial with or parallel to a longitudinal axis of insertion portion 118. Thru hole 188 may be aligned with second lumen 124 of insertion portion 118. The continuous nature of receiving channel 179 and thru hole 188 with second lumen 124 may facilitate smooth insertion of shaft 132 of camera sheath 108 through handle 110 and insertion portion 118, for example, during assembly of medical assembly 102.

To assemble medical assembly 102, camera sheath 108 may be inserted into handle 110 and through second lumen 124. Keying feature 166 and keying surface 168 may align with and be received by receiving features 184 of handle 110. For example, receiving features 184 may at least partially surround keying feature 166. For example, a distal edge of keying feature 166 may abut proximal sides of receiving features 184 (e.g., protrusions or flanges). Thus, receiving features 184 may at least partially surround at least a distal portion of keying feature 166. The protrusions or flanges may inhibit keying feature 166 from advancing further distally. In some aspects, receiving features 184 may be received within one or more grooves surrounding a surface of keying feature 166. This configuration may ensure proper orientation and secure attachment of camera sheath 108 to handle 110 during medical procedures. In aspects, the alignment of keying feature 166 and keying surface 168 with receiving features 184 of handle 110 may prevent rotation of camera sheath 108.

It will be apparent to those skilled in the art that various modifications and variations can be made to the disclosed device without departing from the scope of the disclosure.

For example, medical assemblies described herein may include a medical assembly 102 comprising a medical device 109 and a removable camera. The removable camera may be configured to extend through at least a portion of and couple to medical device 109. In some cases, camera sheath may include keying features and/or surfaces that engage with receiving features 184 of handle 110 to ensure proper alignment during assembly. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

1. A medical system, comprising:
a medical device having a handle and an insertion portion extending from the handle, the insertion portion having a first lumen and a second lumen;
a removable camera sheath configured to removably extend through the second lumen, the removable camera sheath comprising:
a shaft having a proximal end and a distal end, the distal end having an imaging device and a lighting element; and
a connector assembly coupled to the proximal end of the shaft, the connector assembly comprising a keying feature configured to engage with a receiving feature of the handle; and
a needle extending through the first lumen.

2. The medical system of claim 1, wherein the keying feature extends away from a central longitudinal axis of the connector assembly.

3. The medical system of claim 1, wherein the handle comprises a receiving channel configured to receive the keying feature.

4. The medical system of claim 3, wherein the receiving channel comprises a recessed portion having a complementary shape of the keying feature, wherein the receiving channel is configured to accommodate the keying feature.

5. The medical system of claim 1, wherein the connector assembly comprises a keying surface adjacent to the keying feature.

6. The medical system of claim 5, wherein the handle comprises a complementary surface configured to engage with the keying surface.

7. The medical system of claim 6, wherein the connector assembly comprises a casing having a first casing portion and a second casing portion.

8. The medical system of claim 7, wherein the connector assembly further comprises a strain relief disposed between the casing and the shaft.

9. The medical system of claim 8, wherein the strain relief comprises a rib configured to engage with a cutout of the shaft.

10. The medical system of claim 4, wherein the receiving channel includes one or more protrusions or flanges extending laterally inward from a surface of the receiving channel, wherein the one or more flanges are shaped to receive the keying feature of the connector assembly.

11. The medical system of claim 10, wherein the one or more flanges are configured to at least partially surround the keying feature.

12. The medical system of claim 1, wherein a distal end of the removable camera sheath is angled by at least forty degrees relative to a longitudinal axis of the removable camera sheath.

13. The medical system of claim 12, wherein the insertion portion includes a distal opening disposed on a radially outer surface of the insertion portion, wherein the needle is configured to extend through the distal opening, wherein the distal end of the removable camera sheath terminates proximally of the distal opening.

14. The medical system of claim 1, further comprising a controller and a connecting element configured to electronically couple the removable camera sheath to the controller, wherein the connector assembly comprises a receiver configured to removably receive the connecting element.

15. The medical system of claim 14, wherein the connector assembly further comprises a board electrically coupled to the imaging device and the lighting element, the board being configured to transmit data between the imaging device and the controller via the connecting element.

16. A medical system, comprising:
a medical device having a handle and an insertion portion extending from the handle, the insertion portion having at least a first lumen and a second lumen, and the handle defining a receiving channel; and
a removable camera sheath configured to removably extend through the second lumen, the removable camera sheath comprising:
a shaft having a proximal end and a distal end, the distal end having an imaging device and a lighting element; and
a connector assembly coupled to the proximal end of the shaft, the connector assembly comprising a keying feature,
wherein the receiving channel includes one or more flanges extending laterally inward from a surface of the receiving channel, and wherein the one or more flanges are configured to receive the keying feature of the connector assembly.

17. The medical system of claim 16, wherein the one or more flanges are configured to at least partially surround the keying feature.

18. The medical system of claim 17, wherein the connector assembly comprises a strain relief having a rib, and wherein the shaft comprises a cutout configured to receive the rib to maintain proper orientation of the shaft relative to the strain relief, and wherein the rib is integrally formed with the strain relief and extends radially inward from an inner surface of the strain relief.

19. A camera sheath configured for insertion and removal from a medical device, the camera sheath comprising:
a shaft having a proximal end and a distal end, the distal end having an imaging device and a lighting element; and
a connector assembly coupled to the proximal end of the shaft, the connector assembly comprising a keying feature configured to engage with a receiving feature of the medical device, wherein the keying feature includes a cylindrical body extending away from a longitudinal axis of the connector assembly.

20. The camera sheath of claim 19, wherein the distal end of the shaft is angled by at least forty degrees relative to a longitudinal axis of the shaft.
